(19) United States
(12) Patent Application Publication
(10) Pub. No.: US 2025/0075166 A1
(43) Pub. Date: Mar. 6, 2025

(54) ALGAE HARVESTING DEVICE AND METHODS OF USE

(71) Applicant: Chase Leffers, San Francisco, CA (US)

(72) Inventor: Chase Leffers, San Francisco, CA (US)

(21) Appl. No.: 18/820,476

(22) Filed: Aug. 30, 2024

Related U.S. Application Data
(60) Provisional application No. 63/536,807, filed on Sep. 6, 2023.

(51) Int. Cl. C12M 1/00; B01D 39/16; C12M 1/06; C12M 1/12; C12M 1/26

(52) U.S. Cl. CPC C12M 47/02; B01D 39/163; C12M 25/02; C12M 27/02; C12M 33/14

(57) ABSTRACT

The present disclosure provides, among other things, devices and methods for harvesting algae (e.g., microalgae) from algae culture growth media to produce a dried algae product and purified water.

### CROSS REFERENCE TO RELATED APPLICATIONS

This application claims priority from U.S. Provisional Application No. 63/536,807 filed on Sep. 6, 2023, which is incorporated by reference herein in its entirety.

### BACKGROUND

Despite humanity's billions of acres of industrial farmland, endless fleets of cargo planes, and cutting-edge agricultural monocrop science, global food production and distribution remain poorly balanced. There remains a need for devices and methods that provide decentralized access to nutritionally complete food.

### SUMMARY

Devices and methods that advance cultivation and harvesting of locally grown algae offer the prospect of combating malnutrition. The present disclosure provides, among other things, devices and methods for harvesting locally cultivated algae.

In one aspect, this disclosure provides an algae harvesting device comprising a) a housing comprising a separation chamber comprising an inlet, an outlet, and a filter column operably coupled to a mixing element, b) a fluidic shaft, and c) a base configured to concentrically align the separation chamber, the filter column, the mixing element, and the fluidic shaft, wherein the filter column and mixing element are configured to separate algae from an algae growth medium.

In some embodiments, the housing is hermetically sealable. In some embodiments, the filter column comprises a first channel extending longitudinally through the length of the filter column. In some embodiments, the fluidic shaft comprises a second channel extending longitudinally through the length of the fluidic shaft. In some embodiments, the first channel is configured to be in fluid communication with the second channel. In some embodiments, the base comprises a third channel configured to fluidly communicate the growth medium outlet to the second channel.

In some embodiments, the inlet, the separation chamber, the first channel, and the outlet in part (a) are in fluid communication. In some embodiments, the filter column comprises a first end at a top end of the separation chamber and a second end at a bottom end of the separation chamber. In some embodiments, the second end of the filter column is configured to be removably coupled to a top end of the fluidic shaft by a filter adapter.

In some embodiments, the fluidic shaft comprises a bottom end configured to be removably coupled to the base. In some embodiments, the mixing element comprises a plurality of blades coupled to the filter column. In some embodiments, the filter column comprises a porosity of from about 0.1 to about 0.8. In some embodiments, the filter column comprises a pore size of from about 1 μm and 10 km. In some embodiments, the separation chamber comprises a volume of from about 500 ml to about 5000 ml. In some embodiments, the filter column, the mixing element, and the fluidic shaft, are configured to concentrically rotate around a longitudinal axis extending from a top end of the housing to the bottom end of the housing.

In some embodiments, the base further comprises a motor. In some embodiments, the motor is configured to drive the rotation around the longitudinal axis of the filter column, the mixing element, and the fluidic shaft. In some embodiments, the motor comprises an electric motor. In some embodiments, the electric motor comprises a stepper motor, a DC motor, a servomotor. In some embodiments, the stepper motor is coupled to the rotation shaft via a belt.

In some embodiments, the separation chamber further comprises from 1 to 25 inlets. In some embodiments, the separation chamber further comprises from 1 to 25 outlets. In some embodiments, the base comprises from 1 to 25 growth medium outlet. In some embodiments, the wherein the filter column, the mixing element, and the fluidic shaft, are configured to concentrically rotate around the longitudinal axis in a clockwise direction and/or in a counterclockwise direction.

In some embodiments, the device is configured to harvest algae with a yield from about 0.1 g to about 1000 g per day. In some embodiments, the plurality of blades is configured to form a helical shape around the filter column. In some embodiments, the plurality of blades is configured to a) drive the algae growth medium from an algae culture suspension through the filter column, through the first channel, through the second channel, through the third channel, and through the algae growth medium outlet, and b) drive algae separated from the algae culture suspension from the separation chamber to the outlet.

In some embodiments, the device is configured to consume power from an off-grid power source. In some embodiments, the filter column comprises a sintered filter.

In another aspect, the disclosure provides a method of harvesting algae comprising: a) providing an algae harvesting device as herein disclosed, b) driving an algae culture suspension into the inlet of the separation chamber, c) rotating the fluidic shaft connected to the filter column and the mixing element using the motor, d) driving the algae growth medium: i) through the filter column and into the first channel, ii) through the second channel, iv) through the third channel, and iii) through the growth medium outlet, and e) driving the algae through the outlet.

In some embodiments, harvesting is performed substantially continuously for up to 45 days. In some embodiments, harvesting is performed continuously for up to 30 days. In some embodiments, step b) comprises driving the algae culture suspension using a positive displacement pump. In some embodiments, step b) comprises driving the algae culture suspension using a peristaltic pump connected to the growth medium outlet.

In some embodiments, the growth medium is sanitized after passing through the growth medium outlet using ultraviolet sterilization and/or shear stress. In some embodiments, step e) comprises using a peristaltic pump connected to the outlet and collecting the algae in a collection chamber. In some embodiments, step c) comprises rotating in a clockwise direction. In some embodiments, step c) comprises rotating in a counterclockwise direction.

In some embodiments, the method comprises rotating in a substantially reverse direction to clean algae accumulated on the filter column. In some embodiments, step c) comprises rotating at from about 0.1 rpm to about 1000 rpm. In some embodiments, the method comprises harvesting up to 1 kg of algae per day. In some embodiments, the method consumes from about 0 watts to about 500 watts (e.g., about 0 watts to about 400 watts, about 0 watts to about 300 watts, about 0 watts to about 200 watts, about 0 watts to about 100 watts, about 0 watts to about 50 watts, about 0 watts to about 40 watts, about 0 watts to about 30 watts, about 0 watts to about 20 watts, about 0 watts to about 10 watts, about 0 watts to about 5 watts, about 1 watts to about 500 watts, about 5 watts to about 500 watts, about 10 watts to about 500 watts, about 50 watts to about 500 watts, about 100 watts to about 500 watts, about 200 watts to about 500 watts, about 300 watts to about 500 watts, about 400 watts to about 500 watts, about 450 watts to about 500 watts, about 0 watts, about 1 watts, about 2 watts, about 3 watts, about 4 watts, about 5 watts, about 6 watts, about 7 watts, about 8 watts, about 9 watts, about 10 watts, about 25 watts, about 50 watts, about 75 watts, about 100 watts, about 125 watts, about 150 watts, about 175 watts, about 200 watts, about 225 watts, about 250 watts, about 275 watts, about 300 watts, about 325 watts, about 350 watts, about 375 watts, about 400 watts, about 425 watts, about 450 watts, about 475 watts, or about 500 watts) of power per hour.

### Definitions

A, An, The, Or: As used herein, “a”, “an”, and “the” refer to one or to more than one (i.e., to at least one) of the grammatical object of the article. By way of example, “an element” discloses embodiments of exactly one element and embodiments including more than one element. As used herein, the terms “or” and “and/or”, as conjunctions in a list of at least two elements, encompass and disclose embodiments in which the listed elements are included in the alternative, together, or in any combination.

About: As used herein, term “about”, when used in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referenced value.

Algae: As used herein, the term “algae” refers to any photosynthetic microorganism including, but not limited to, prokaryotic algae, eukaryotic algae, yeast, and/or other single-celled microorganisms.

Algae growth medium: As used herein, the term “algae growth medium” or “algae medium” or “culture medium” refers to a fluid containing the nutrients and dissolved gases that are necessary for the growth of viable algae in culture. In some embodiments, the algae growth medium comprises fertilizers including nitrogen, phosphorus, and/or potassium. In some embodiments, the algae growth medium comprises trace amounts of metals e.g., iron and zinc.

Concentrically aligned: As used herein, the term “concentrically align,” or “concentrical alignment,” or “concentrically aligned,” or variants thereof, refers to a configuration of multiple components of the device herein described (e.g., the separation chamber, the filter column, the mixing element, and/or the fluidic shaft, among others) where the components have the same center which forms an axis about which the components may rotate.

Coupled: As used herein, the term “coupled,” refers to the joining of two or more components directly or indirectly to one another. Such joining may be stationary in nature or movable in nature. In some embodiments, such joining may be achieved with the components (or the components and any additional intermediate component) being attached to one another. In some embodiments, such joining may be achieved with the components (or the components and any additional intermediate components) being integrally formed as a single unitary body with one another. Such joining may be permanent in nature or alternatively may be removable or releasable in nature. In some embodiments, the term “coupled” is interchangeable with the term “operably coupled,” which refers to two or more components being directly or indirectly joined such that motion may be transmitted from one component to the other component directly or via intermediate components.

Fluid communication: As used herein, the term “in fluid communication,” refers to components that are arranged and configured to allow fluid to move therebetween. Two or more parts of the device can be in fluid communication by being physically linked together or adjacent one another, or the fluid communication can be mediated through another part of the device. In some embodiments, the term “in valved fluid communication,” is used herein, and refers to two or more components of the algae harvesting device having a valve disposed between the components, such that upon opening or actuating the valve, fluid communication between the components is established.

Hermetically sealed: As used herein, the term “hermetically sealed,” refers to a complete seal against the escape or entry of external liquid, humidity, and/or air. In some embodiments, the term hermetically sealed illustrates that undesired moisture and/or gases cannot permeate across a surface of a component (e.g., moisture and/or gases cannot permeate the housing and enter the separation chamber) at all or only to a minimal extent.

Off-grid: As used herein, the term “off-grid,” refers to an element that is not directly connected to a system, and/or infrastructure configured for, usually, bulk electric-power transmission of electrical energy, from generating power plants to electrical substations located near demand centers. In some embodiments, the term off-grid refers to the use of a component and/or device without direct connection to an interconnected electrical power network or grid. In some embodiments, the term off-grid refers to consumption of locally generated electrical power (e.g., from a locally positioned power station) such that the consumption is independent from power produced by an interconnected electrical power network or grid.

Sanitized: As used herein, the term “sanitized,” or grammatical variants thereof, refers to a liquid medium substantially free of microalgae organisms.

Separation: As used herein, the term “separate,” or “separation,” or variants thereof, refers to substantially isolating one substance from another. In some embodiments, separation is used to describe the isolation of algae form the algae growth medium to obtain a product enriched in algae and a growth medium no longer in contact with the algae.

### BRIEF DESCRIPTION OF THE DRAWINGS

FIG. 1 is a schematic drawing showing a cross-sectional side view of an exemplary algae harvesting device. The housing 100, separation chamber 200, inlet 210, filter column 300, mixing element 400, fluidic shaft 500, fluid lock 550, base 600, and motor 650 are shown.

FIG. 2 is a schematic drawing showing a cross-sectional side view of the exemplary algae harvesting device. The housing 100, separation chamber 200, outlet 220, filter column 300, first channel 310, mixing element 400, fluidic shaft 500, second channel 510, fluid lock 550, base 600, third channel 610, growth medium outlet 620, and motor 650 are shown.

FIG. 3 is a schematic drawing showing a cross-sectional side view of the exemplary algae harvesting device. The housing 100, separation chamber 200, outlet 220, mixing element 400, fluidic shaft 500, fluid lock 550, base 600, and motor 650 are shown.

FIG. 4 is a schematic drawing showing a side view of filter column 300 and mixing element 400 inside separation chamber 200. The first end 302 of the filter column 300, the second end 304 of the filter column 300, the top end 230 of separation chamber 200, the bottom end 240 of separation chamber 200 are also shown.

FIG. 5 is a schematic drawing showing a cross-sectional side view of filter column 300, first channel 310 and mixing element 400 inside separation chamber 200. The first end 302 of the filter column 300, the second end 304 of the filter column 300, the top end 230 of separation chamber 200, the bottom end 240 of separation chamber 200 are also shown.

FIG. 6 is a schematic drawing showing a cross-sectional side view of second end 304 of filter column 300, first channel 310, fluidic shaft 500, top end of fluidic shaft 502, bottom end of fluidic shaft 504, second channel 510, base 600, third channel 610, growth medium outlet 620, and motor 650.

### DETAILED DESCRIPTION

The algae harvesting device and methods of use thereof herein provided are configured to produce consumable algae (e.g., algae for nutritional consumption) in an autonomous, low-maintenance, low-power consuming approach that is amenable to consumer-level use and is scalable to industrial-size algae production and harvesting.

### Algae Harvesting Device

In some embodiments, the algae harvesting device is a part of an algae production system. In some embodiments, the algae production system comprises, among other things, algae cultivation components, power source(s), and growth media processing components. In some embodiments, the algae harvesting device herein disclosed is configured to receive an algae culture suspension, continuously or periodically, separate the algae culture suspension into its two major constituents, algae and algae growth medium, and separately send the algae and the algae growth medium for further processing to other components of an algae production system.

In some embodiments, the harvesting device herein disclosed is configured to harvest algae with a yield of from about 0.1 g to about 1,000 g (e.g., from about 0.5 g to about 1,000 g, from about 1 g to about 1,000 g, from about 10 g to about 1,000 g, from about 100 g to about 500 g, from about 100 g to about 250 g, about 1 g, about 5 g, about 10 g, about 15 g, about 20 g, about 25 g, about 30 g, about 35 g, about 40 g, about 45 g, about 50 g, about 55 g, about 60 g, about 65 g, about 70 g, about 75 g, about 80 g, about 85 g, about 90 g, about 95 g, about 100 g, about 105 g, about 110 g, about 115 g, about 120 g, about 125 g, about 130 g, about 135 g, about 140 g, about 145 g, about 150 g, about 155 g, about 160 g, about 165 g, about 170 g, about 175 g, about 180 g, about 185 g, about 190 g, about 195 g, about 200 g, about 205 g, about 210 g, about 215 g, about 220 g, about 225 g, about 230 g, about 235 g, about 240 g, about 245 g, about 250 g, about 255 g, about 260 g, about 265 g, about 270 g, about 275 g, about 280 g, about 285 g, about 290 g, about 295 g, about 300 g, about 305 g, about 310 g, about 315 g, about 320 g, about 325 g, about 330 g, about 335 g, about 340 g, about 345 g, about 350 g, about 355 g, about 360 g, about 365 g, about 370 g, about 375 g, about 380 g, about 385 g, about 390 g, about 395 g, about 400 g, about 405 g, about 410 g, about 415 g, about 420 g, about 425 g, about 430 g, about 435 g, about 440 g, about 445 g, about 450 g, about 455 g, about 460 g, about 465 g, about 470 g, about 475 g, about 480 g, about 485 g, about 490 g, about 495 g, about 500 g, about 505 g, about 510 g, about 515 g, about 520 g, about 525 g, about 530 g, about 535 g, about 540 g, about 545 g, about 550 g, about 555 g, about 560 g, about 565 g, about 570 g, about 575 g, about 580 g, about 585 g, about 590 g, about 595 g, about 600 g, about 605 g, about 610 g, about 615 g, about 620 g, about 625 g, about 630 g, about 635 g, about 640 g, about 645 g, about 650 g, about 655 g, about 660 g, about 665 g, about 670 g, about 675 g, about 680 g, about 685 g, about 690 g, about 695 g, about 700 g, about 705 g, about 710 g, about 715 g, about 720 g, about 725 g, about 730 g, about 735 g, about 740 g, about 745 g, about 750 g, about 755 g, about 760 g, about 765 g, about 770 g, about 775 g, about 780 g, about 785 g, about 790 g, about 795 g, about 800 g, about 805 g, about 810 g, about 815 g, about 820 g, about 825 g, about 830 g, about 835 g, about 840 g, about 845 g, about 850 g, about 855 g, about 860 g, about 865 g, about 870 g, about 875 g, about 880 g, about 885 g, about 890 g, about 895 g, about 900 g, about 905 g, about 910 g, about 915 g, about 920 g, about 925 g, about 930 g, about 935 g, about 940 g, about 945 g, about 950 g, about 955 g, about 960 g, about 965 g, about 970 g, about 975 g, about 980 g, about 985 g, about 990 g, about 995 g, or about 1000 g) per day.

In some embodiments, the algae harvesting device is configured to consume power from an off-grid power source. In some embodiments, the algae harvesting device is configured to consume electrical power from an off-grid power source. In some embodiments, the algae harvesting device is configured to consume electrical power from a solar panel and/or solar panel array. In some embodiments, the algae harvesting device is configured to consume electrical power from a wind power source, a hydropower source, external generators (e.g., gas, or diesel consuming generators), or human mechanical power (e.g., a bicycle). In some embodiments, the algae harvesting device is configured to consume from about 0 watts to about 500 watts (e.g., about 0 watts to about 400 watts, about 0 watts to about 300 watts, about 0 watts to about 200 watts, about 0 watts to about 100 watts, about 0 watts to about 50 watts, about 0 watts to about 40 watts, about 0 watts to about 30 watts, about 0 watts to about 20 watts, about 0 watts to about 10 watts, about 0 watts to about 5 watts, about 1 watts to about 500 watts, about 5 watts to about 500 watts, about 10 watts to about 500 watts, about 50 watts to about 500 watts, about 100 watts to about 500 watts, about 200 watts to about 500 watts, about 300 watts to about 500 watts, about 400 watts to about 500 watts, about 450 watts to about 500 watts, about 0 watts, about 1 watts, about 2 watts, about 3 watts, about 4 watts, about 5 watts, about 6 watts, about 7 watts, about 8 watts, about 9 watts, about 10 watts, about 25 watts, about 50 watts, about 75 watts, about 100 watts, about 125 watts, about 150 watts, about 175 watts, about 200 watts, about 225 watts, about 250 watts, about 275 watts, about 300 watts, about 325 watts, about 350 watts, about 375 watts, about 400 watts, about 425 watts, about 450 watts, about 475 watts, or about 500 watts) of power per hour.

In some embodiments, the algae harvesting device herein described includes housing 100 comprising separation chamber 200, fluidic shaft 500, and base 600.

### Housing 100 and Separation Chamber 200

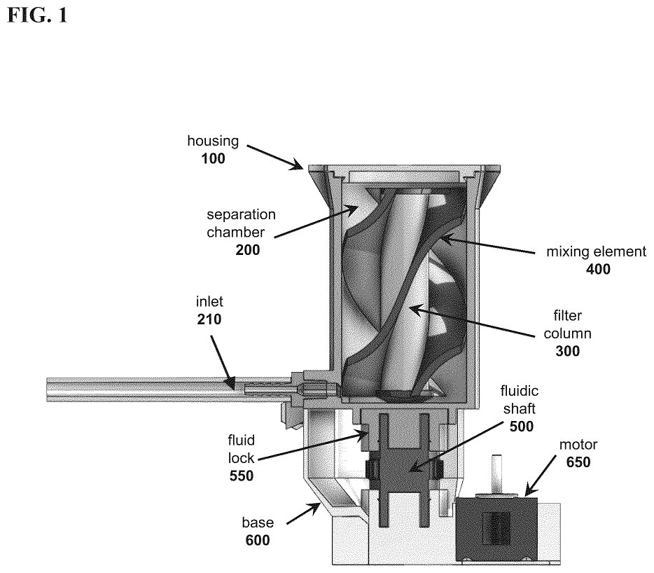

In some embodiments, the algae harvesting device comprises housing 100. In some embodiments, housing 100 is a hollow vessel comprising separation chamber 200 (FIG. 1). In some embodiments, housing 100 has a substantially cylindrical shape. In some embodiments, housing 100 comprises a top end and a bottom end.

In some embodiments, housing 100 is composed of polylactic acid (PLA), acrylonitrile butadiene styrene (ABS), polycarbonate, acrylic (e.g., poly(methyl methacrylate), polystyrene, polypropylene, or aluminum.

In some embodiments, housing 100 comprises one or more fluid ports that fluidically communicate separation chamber 200 with one or more algae culture suspension sources and with one or more algae collection compartments. In some embodiments, housing 100 comprises a first plurality, from 1 to 25 (e.g., 1, 2, 3, 4, 5, 6, 7, 8, 9, 10, 11, 12, 13, 14, 15, 16, 17, 18, 19, 20, 21, 22, 23, 24, or 25), of openings that fluidly communicate one or more (e.g., 1, 2, 3, 4, 5, 6, 7, 8, 9, or 10) algae-lade fluid sources to separation chamber 200 via the inlet(s) of separation chamber 200. In some embodiments, housing 100 comprises a second plurality, from 1 to 25 (e.g., 1, 2, 3, 4, 5, 6, 7, 8, 9, 10, 11, 12, 13, 14, 15, 16, 17, 18, 19, 20, 21, 22, 23, 24, or 25), of openings that that fluidly communicate one or more (e.g., 1, 2, 3, 4, 5, 6, 7, 8, 9, or 10) algae-lade fluid sources to separation chamber 200 via the outlet(s) of separation chamber 200.

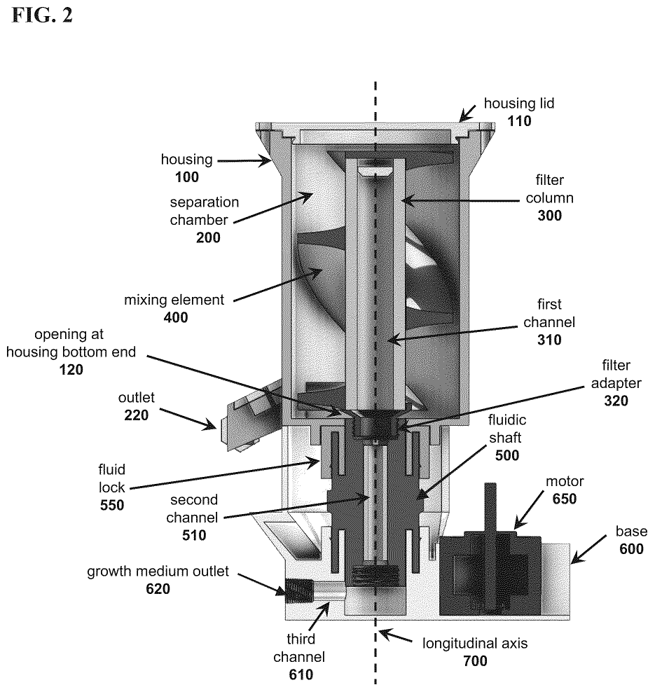

In some embodiments, housing 100 is configured to be hermetically sealable. In some embodiments, the bottom end of housing 100 is configured to couple with filter adapter 320, fluidic shaft 500, fluid lock 550, and/or base 600 to hermetically seal separation chamber 200 to moisture and/or gasses from entering and/or exiting separation chamber 200 via routes that are not inlet 210, outlet 220, first channel 310, second channel 510, third channel 610, or growth medium outlet 620. In some embodiments, the top end of housing 100 comprises a housing lid 110 (FIG. 2). In some embodiments, housing lid 110 is separable from housing 100. In some embodiments, housing lid is integral to housing 100. In some embodiments, housing lid 110 is configured to couple to the top end of housing 100 to hermetically seal housing 100 to moisture and/or gasses from entering and/or exiting separation chamber 200 via routes that are not inlet 210, outlet 220, first channel 310, second channel 510, third channel 610, or growth medium outlet 620. Any known methods useful for hermetically sealing may be employed including the use of threads, LOCTITE®, epoxies, liquid shim, or an O-ring.

In some embodiments, the bottom end of housing 100 comprises opening 120 (FIG. 2). In some embodiments, opening 120 comprises a diameter of from about 5 mm to about 50 mm (e.g., about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, or about 50 mm). In some embodiments, opening 120 is configured to, at least partially, couple and/or support filter adapter 320 (FIG. 2).

In some embodiments, housing 100 and separation chamber 200 are configured to concentrically align with filter column 300, filter adapter 320, mixing element 400, fluidic shaft 500, and second channel 510 (FIG. 2).

In some embodiments, separation chamber 200, comprises a top end and a bottom end. In some embodiments, separation chamber 200 comprises from 1 to 25 (e.g., 1, 2, 3, 4, 5, 6, 7, 8, 9, 10, 11, 12, 13, 14, 15, 16, 17, 18, 19, 20, 21, 22, 23, 24, or 25) inlet(s) 210. In some embodiments, separation chamber 200 comprises from 1 to 10 (e.g., 1, 2, 3, 4, 5, 6, 7, 8, 9, or 10) inlet(s) 210. In some embodiments, separation chamber 200 comprises from 1 to 5 (e.g., 1, 2, 3, 4, or 5) inlet(s) 210. In some embodiments, separation chamber 200 comprises from 1 to 25 (e.g., 1, 2, 3, 4, 5, 6, 7, 8, 9, 10, 11, 12, 13, 14, 15, 16, 17, 18, 19, 20, 21, 22, 23, 24, or 25) outlet(s) 220. In some embodiments, separation chamber 200 comprises from 1 to 10 (e.g., 1, 2, 3, 4, 5, 6, 7, 8, 9, or 10) outlet(s) 220. In some embodiments, separation chamber 200 comprises from 1 to 5 (e.g., 1, 2, 3, 4, or 5) outlet(s) 220. In some embodiments, separation chamber 200 further comprises filter column 300 operably coupled to mixing element 400. In some embodiments, separation chamber 200 is in fluid communication with inlet 210, outlet 220, first channel 310, second channel 510, third channel 610, and growth medium outlet 620. In some embodiments, separation chamber 200 is in valved fluid communication with inlet 210, outlet 220, first channel 310, second channel 510, third channel 610, and growth medium outlet 620.

In some embodiments, separation chamber 200 comprises a volume of from about 500 ml to about 5000 ml (e.g., about 500 ml, about 600 ml, about 700 ml, about 800 ml, about 900 ml, about 1000 ml, about 1100 ml, about 1200 ml, about 1300 ml, about 1400 ml, about 1500 ml, about 1600 ml, about 1700 ml, about 1800 ml, about 1900 ml, about 2000 ml, about 2100 ml, about 2200 ml, about 2300 ml, about 2400 ml, about 2500 ml, about 2600 ml, about 2700 ml, about 2800 ml, about 2900 ml, about 3000 ml, about 3100 ml, about 3200 ml, about 3300 ml, about 3400 ml, about 3500 ml, about 3600 ml, about 3700 ml, about 3800 ml, about 3900 ml, about 4000 ml, about 4100 ml, about 4200 ml, about 4300 ml, about 4400 ml, about 4500 ml, about 4600 ml, about 4700 ml, about 4800 ml, about 4900 ml, or about 5000 ml).

In some embodiments, separation chamber 200 comprises filter column 300. In some embodiments, separation chamber 200 comprises filter column 300 operably coupled to mixing element 400 (FIG. 2).

### Filter Column 300

The algae harvesting device herein described may include filter column 300 (FIG. 1 and FIG. 2). In some embodiments, filter column 300 comprises a first end 302 and a second end 304. In some embodiments, first end 302 is configured to be disposed adjacent to the top end of separation chamber 230. In some embodiments, second end 304 is configured to be disposed adjacent to the bottom end of separation chamber 240.

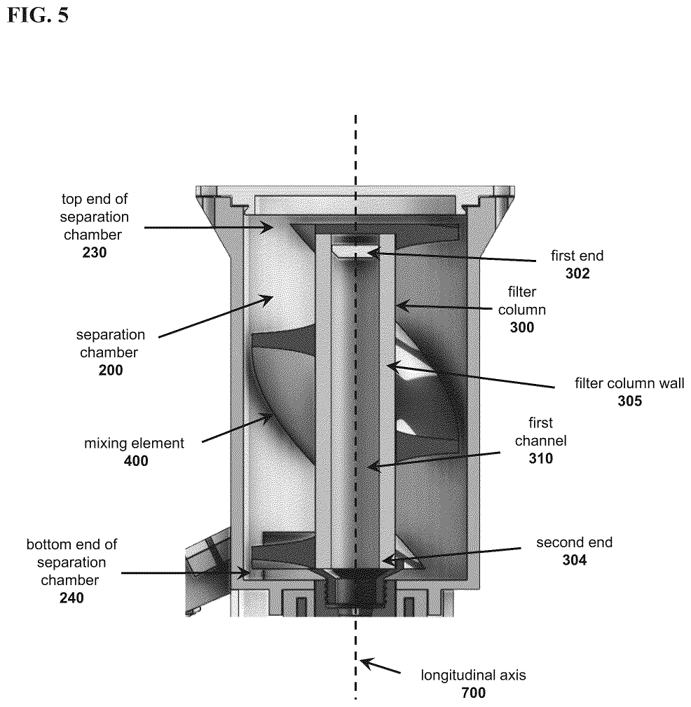

In some embodiments, filter column 300 has a substantially cylindrical shape. In some embodiments, filter column 300 comprises a first channel 310 that extends longitudinally through the length of filter column 300. In some embodiments, filter column 300 comprises a first channel 310 that extends from first end 302 to second end 304 of filter column 300. In some embodiments, filter column 300 is a cylindrical filter comprising a first channel 310 that extends the entire length of filter column 300 from first end 302 to second end 304 (FIG. 5). In some embodiments, first channel 310 is configured to be in fluid communication with second channel 510. In some embodiments, first channel 310 is configured to be in valved fluid communication with second channel 510.

In some embodiments, filter column 300 has a length from first end 302 to second end 304 of from about 5 cm to about 100 cm (e.g., from about 5 cm to about 90 cm, from about 5 cm to about 80 cm, from about 5 cm to about 70 cm, from about 5 cm to about 60 cm, from about 5 cm to about 50 m, from about 5 cm to about 40 cm, from about 5 cm to about 30 cm, from about 5 cm to about 20 cm, from about 5 cm to about 10 cm, from about 10 cm to about 100 cm, from about 20 cm to about 100 cm, from about 30 cm to about 100 cm, from about 40 cm to about 100 cm, from about 50 cm to about 100 cm, from about 60 cm to about 100 cm, from about 70 cm to about 100 cm, from about 80 cm to about 100 cm, from about 90 cm to about 100 cm, or about 5 cm, about 10 cm, about 15 cm, about 20 cm, about 25 cm, about 30 cm, about 35 cm, about 40 cm, about 45 cm, about 50 cm, about 55 cm, about 60 cm, about 65 cm, about 70 cm, about 75 cm, about 80 cm, about 85 cm, about 90 cm, about 95 cm, or about 100 cm). In some embodiments, the length of filter column 300 extends the entire length from top end of separation chamber 230 to bottom end of separation chamber 240. In some embodiments, the length of filter column 300 extends from about 10% to about 99% (e.g., about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) of the distance from top end of separation chamber 230 to bottom end of separation chamber 240.

In some embodiments, filter column 300 comprises an inner diameter and an outer diameter. In some embodiments, the inner diameter is the diameter of first channel 310. In some embodiments, first channel 310 comprises a diameter of from about 1 cm to about 100 cm (e.g., about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 31 cm, about 32 cm, about 33 cm, about 34 cm, about 35 cm, about 36 cm, about 37 cm, about 38 cm, about 39 cm, about 40 cm, about 41 cm, about 42 cm, about 43 cm, about 44 cm, about 45 cm, about 46 cm, about 47 cm, about 48 cm, about 49 cm, about 50 cm, about 51 cm, about 52 cm, about 53 cm, about 54 cm, about 55 cm, about 56 cm, about 57 cm, about 58 cm, about 59 cm, about 60 cm, about 61 cm, about 62 cm, about 63 cm, about 64 cm, about 65 cm, about 66 cm, about 67 cm, about 68 cm, about 69 cm, about 70 cm, about 71 cm, about 72 cm, about 73 cm, about 74 cm, about 75 cm, about 76 cm, about 77 cm, about 78 cm, about 79 cm, about 80 cm, about 81 cm, about 82 cm, about 83 cm, about 84 cm, about 85 cm, about 86 cm, about 87 cm, about 88 cm, about 89 cm, about 90 cm, about 91 cm, about 92 cm, about 93 cm, about 94 cm, about 95 cm, about 96 cm, about 97 cm, about 98 cm, about 99 cm, or about 100 cm). In some embodiments, filter column comprises a column wall comprising a filtering material. In some embodiments, the filtering material comprises a metal, a metal alloy, a glass, a ceramic, sintered metal (e.g., brass, aluminum, bronze, steel, or any alloys thereof), plastic (e.g., PLA or acrylic), ultraviolet dried resin, fabric, or composite materials. In some embodiments filter column 300 is a sintered filter. In some embodiments, filter column 300 is a composite fabric filter (e.g., a fabric wrapped around a solid material). In some embodiments, filter column 300 is a 3D printed filter. In some embodiments, the filter material has a thickness of from about 0.1 mm to about 20 mm (e.g., about 0.1 mm, about 0.2 mm, about 0.3 mm, about 0.4 mm, about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, or about 20 mm).

In some embodiments, the outer diameter of filter column 300 includes the inner diameter and twice the thickness of the filter material. In some embodiments, the outer diameter is from about 1 cm to about 104 cm (e.g., about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 31 cm, about 32 cm, about 33 cm, about 34 cm, about 35 cm, about 36 cm, about 37 cm, about 38 cm, about 39 cm, about 40 cm, about 41 cm, about 42 cm, about 43 cm, about 44 cm, about 45 cm, about 46 cm, about 47 cm, about 48 cm, about 49 cm, about 50 cm, about 51 cm, about 52 cm, about 53 cm, about 54 cm, about 55 cm, about 56 cm, about 57 cm, about 58 cm, about 59 cm, about 60 cm, about 61 cm, about 62 cm, about 63 cm, about 64 cm, about 65 cm, about 66 cm, about 67 cm, about 68 cm, about 69 cm, about 70 cm, about 71 cm, about 72 cm, about 73 cm, about 74 cm, about 75 cm, about 76 cm, about 77 cm, about 78 cm, about 79 cm, about 80 cm, about 81 cm, about 82 cm, about 83 cm, about 84 cm, about 85 cm, about 86 cm, about 87 cm, about 88 cm, about 89 cm, about 90 cm, about 91 cm, about 92 cm, about 93 cm, about 94 cm, about 95 cm, about 96 cm, about 97 cm, about 98 cm, about 99 cm, about 100 cm, about 101 cm, about 102 cm, about 103 cm, or about 104 cm).

In some embodiments, filter column 300 comprises a pore size of from about 1 μm to about 10 μm (e.g., from about 1 μm to about 9.5 μm, from about 1 μm to about 9 μm, from about 1 μm to about 8.5 μm, from about 1 μm to about 8 μm, from about 1 μm to about 7.5 μm, from about 1 μm to about 7 μm, from about 1 μm to about 6.5 μm, from about 1 μm to about 6 μm, from about 1 μm to about 5.5 μm, from about 1 μm to about 5 μm, from about 1 μm to about 4.5 μm, from about 1 μm to about 4 μm, from about 1 μm to about 3.5 am, from about 1 μm to about 3 μm, from about 1 μm to about 2.5 μm, from about 1 μm to about 2 am, from about 1 μm to about 1.5 μm, from about 1.5 μm to about 10 am, from about 2 μm to about 10 μm, from about 2.5 μm to about 10 μm, from about 3 μm to about 10 μm, from about 3.5 μm to about 10 μm, from about 4 μm to about 10 μm, from about 4.5 μm to about 10 μm, from about 5 μm to about 10 μm, from about 5.5 μm to about 10 μm, from about 6 μm to about 10 μm, from about 6.5 μm to about 10 μm, from about 7 μm to about 10 μm, from about 7.5 μm to about 10 μm, from about 8 μm to about 10 μm, from about 8.5 μm to about 10 μm, from about 9 μm to about 10 μm, from about 9.5 μm to about 10 μm, about 2 μm, about 3 μm, about 4 μm, about 5 μm, about 6 μm, about 7 μm, about 8 μm, about 9 μm, or about 10 μm).

In some embodiments, filter column 300 comprises a porosity of from about 0.1 to about 0.8 (e.g., from about 0.1 to about 0.7, from about 0.1 to about 0.6, from about 0.1 to about 0.5, from about 0.1 to about 0.4, from about 0.1 to about 0.3, from about 0.1 to about 0.2, from about 0.2 to about 0.7, from about 0.3 to about 0.7, from about 0.4 to about 0.7, from about 0.5 to about 0.7, from about 0.6 to about 0.7, from about 0.2 to about 0.6, from about 0.3 to about 0.5, about 0.1, about 0.2, about 0.3, about 0.4, about 0.5, about 0.6, about 0.7, or about 0.8).

In some embodiments, filter column 300 and mixing element 400 are operably coupled. In some embodiments, filter column 300 and mixing element 400 are separable. In some embodiments, filter column 300 and mixing element 400 are integral. In some embodiments, mixing element 400 is absent. In some embodiments, mixing element 400 and filter column 300 can rotate relative to each other. In some embodiments, filter column 300 is configured to rotate and mixing element 400 is configured to slip around rotating filter column 300, causing friction, which spins mixing element 400. In some embodiments, mixing element 400 is attached (e.g., epoxied, glued, and/or welded) to filter column 300 to the filter column.

In some embodiments, filter column 300 and mixing element 400 share a common center along a longitudinal axis 700 extending from top end of separation chamber 230 to bottom end of separation chamber 240 (i.e., filter column 300 and mixing element 400 are concentrically aligned with each other) (FIG. 5). In some embodiments, filter column 300 and mixing element 400 are configured to rotate around longitudinal axis 700. In some embodiments, longitudinal axis 700 is a stationary axis.

### Filter Adapter 320

In some embodiments, second end of filter column 304 is coupled to filter adapter 320 (FIG. 5). In some embodiments, second end of filter column 304 and filter adapter 320 are operably coupled. In some embodiments, second end of filter column 304 and filter adapter 320 are separable. In some embodiments, second end of filter column 304 and filter adapter 320 are integral. In some embodiments, second end of filter column 304 is configured to be fastened (e.g., screwed, snap-fit, press-fit, epoxied, glued, and/or welded) to filter adapter 320. In some embodiments, filter adapter 320 is configured to be fastened (e.g., screwed, snap-fit, press-fit, epoxied, glued, and/or welded) to fluid lock 550.

In some embodiments, filter adapter 320 comprises a first section and a second section. In some embodiments, the first section of filter adapter 320 comprises a conical shape. In some embodiments, the second section of filter adapter 320 comprises a cylindrical shape FIG. 2 and FIG. 5). In some embodiments, the first section of filter adapter 320 couples with filter column 300. In some embodiments, the second section of filter adapter 320 couples with filter column 300. In some embodiments, the first section of filter adapter 320 couples with fluidic shaft 500. In some embodiments, the second section of filter adapter 320 couples with fluidic shaft 500. In some embodiments, the first section of filter adapter 320 couples with filter column 300 and the second section of filter adapter 320 couples with fluidic shaft 500. In some embodiments, the first section of filter adapter 320 couples with fluidic shaft 500 and the second section of filter adapter 320 couples with filter column 300.

In some embodiments, filter adapter 320 is configured to fit inside opening 120 at the bottom end of housing 100. In some embodiments, the first section of filter adapter 320 comprises a diameter of from about 1 cm to about 100 cm (e.g., about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 31 cm, about 32 cm, about 33 cm, about 34 cm, about 35 cm, about 36 cm, about 37 cm, about 38 cm, about 39 cm, about 40 cm, about 41 cm, about 42 cm, about 43 cm, about 44 cm, about 45 cm, about 46 cm, about 47 cm, about 48 cm, about 49 cm, about 50 cm, about 51 cm, about 52 cm, about 53 cm, about 54 cm, about 55 cm, about 56 cm, about 57 cm, about 58 cm, about 59 cm, about 60 cm, about 61 cm, about 62 cm, about 63 cm, about 64 cm, about 65 cm, about 66 cm, about 67 cm, about 68 cm, about 69 cm, about 70 cm, about 71 cm, about 72 cm, about 73 cm, about 74 cm, about 75 cm, about 76 cm, about 77 cm, about 78 cm, about 79 cm, about 80 cm, about 81 cm, about 82 cm, about 83 cm, about 84 cm, about 85 cm, about 86 cm, about 87 cm, about 88 cm, about 89 cm, about 90 cm, about 91 cm, about 92 cm, about 93 cm, about 94 cm, about 95 cm, about 96 cm, about 97 cm, about 98 cm, about 99 cm, or about 100 cm). In some embodiments, the second section of filter adapter 320 comprises a diameter of from about 5 mm to about 50 mm (e.g., about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, or about 50 mm). In some embodiments, filter adapter is concentrically aligned with filter column and fluidic shaft.

In some embodiments, filter adapter 320 is removably coupled to a top end of fluidic shaft 500. In some embodiments, filter column 300 and fluidic shaft 500 are removably coupled by filter adapter 320. In some embodiments, second end 304 of filter column 300 is configured to be removably coupled to top end 502 of fluidic shaft 500. In some embodiments, second end 304 of filter column 300 is configured to be removably coupled to top end 502 of fluidic shaft 500 by filter adapter 320. In some embodiments, second end of filter column 304 is configured to be operably coupled and assembled in fluid communication with top end of fluidic shaft 500 by filter adapter 320. In some embodiments, the filter adapter 320 is configured to operably connect any one of a plurality of filter columns to the top end of the fluidic shaft.

### Mixing Element 400

In some embodiments, separation chamber 200 comprises mixing element 400 operably coupled to filter column 300. In some embodiments, mixing element 400 and filter column 300 are configured to separate algae from an algae growth medium. In some embodiments, mixing element 400 is configured to be concentrically aligned with separation chamber 200, filter column 300, and fluidic shaft 500. In some embodiments, mixing element 400 is configured to be concentrically aligned with separation chamber 200, filter column 300, and fluidic shaft 500 so as to share a common center along longitudinal axis 700 (FIG. 5). In some embodiments, mixing element 400, is configured to rotate around longitudinal axis 700 and the rotation of mixing element 400 drives the separation of algae from the growth culture medium in which the algae is suspended. In some embodiments, mixing element 400 is configured to concentrically rotate around longitudinal axis 700 with filter column 300, fluidic shaft 500, in a clockwise direction or in a counterclockwise direction from any perspective along longitudinal axis 700.

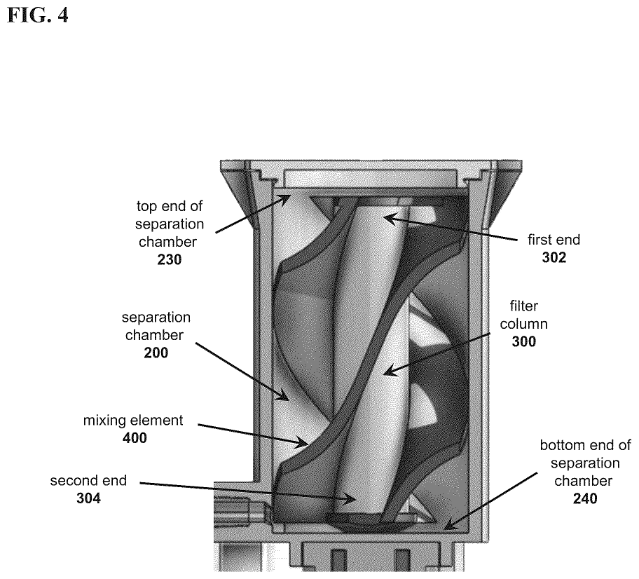

In some embodiments, mixing element 400 comprises a plurality (e.g., 1, 2, 3, 4, 5, 6, 7, 8, 9, or 10) of blades. In some embodiments, mixing element 400 comprises a plurality of blades coupled to filter column 300. In some embodiments, mixing element 400 comprises a plurality of blades that are configured to form a helical shape around filter column 300 (FIG. 4). In some embodiments, mixing element 400 comprises a plurality of blades that are configured to form a helical shape around longitudinal axis 700 (FIG. 5). In some embodiments, mixing element 400 comprises a plurality of blades composed of a substantially rigid material. In some embodiments, mixing element 400 comprises a plurality of blades composed of a substantially flexible material. In some embodiments, mixing element 400 is composed of PLA, ABS, polycarbonate, acrylic, polypropylene, or aluminum.

In some embodiments, the blades of mixing element 400 have a blade width equal to the radius of separation chamber. In some embodiments, the blades of mixing element 400 comprise a blade width of from about 1 cm to about 100 cm (e.g., about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 31 cm, about 32 cm, about 33 cm, about 34 cm, about 35 cm, about 36 cm, about 37 cm, about 38 cm, about 39 cm, about 40 cm, about 41 cm, about 42 cm, about 43 cm, about 44 cm, about 45 cm, about 46 cm, about 47 cm, about 48 cm, about 49 cm, about 50 cm, about 51 cm, about 52 cm, about 53 cm, about 54 cm, about 55 cm, about 56 cm, about 57 cm, about 58 cm, about 59 cm, about 60 cm, about 61 cm, about 62 cm, about 63 cm, about 64 cm, about 65 cm, about 66 cm, about 67 cm, about 68 cm, about 69 cm, about 70 cm, about 71 cm, about 72 cm, about 73 cm, about 74 cm, about 75 cm, about 76 cm, about 77 cm, about 78 cm, about 79 cm, about 80 cm, about 81 cm, about 82 cm, about 83 cm, about 84 cm, about 85 cm, about 86 cm, about 87 cm, about 88 cm, about 89 cm, about 90 cm, about 91 cm, about 92 cm, about 93 cm, about 94 cm, about 95 cm, about 96 cm, about 97 cm, about 98 cm, about 99 cm, or about 100 cm).

In some embodiments, mixing element 400 comprises one or more mixing structures including blades, helical mixers, discs, brushes, woven strings, walls, turbines, and/or turbines.

In some embodiments, the plurality of blades of mixing element 400 is configured to 1) drive the algae growth medium from an algae culture suspension through filter column 300, through first channel 310, through the second channel 510, through third channel 610, and through algae growth medium outlet 620 and 2) drive algae separated from the algae culture suspension from separation chamber 200 to the outlet 220.

### Fluidic Shaft 500

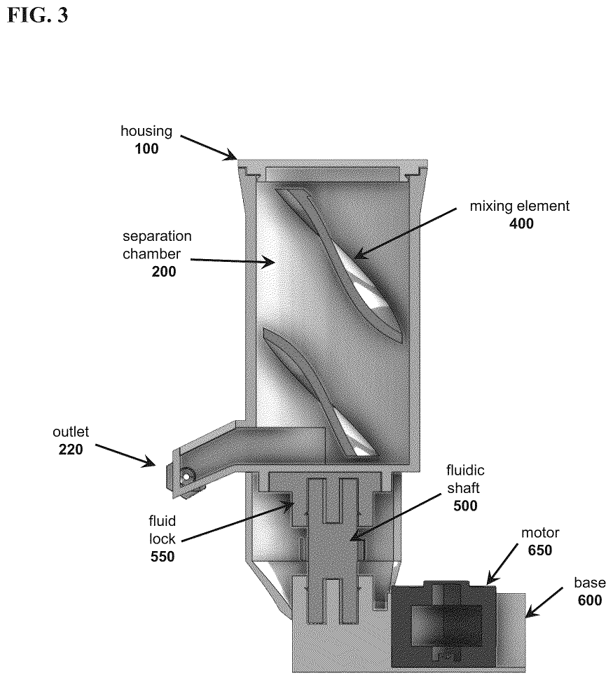

The algae harvesting devices herein disclosed comprise a fluidic shaft 500 (FIGS. 1-3). In some embodiments, fluidic shaft 500 comprises a top end 502. In some embodiments, fluidic shaft 500 comprises a bottom end 504. In some embodiments, fluidic shaft 500 comprises bottom end 504 that is configured to be removably coupled to base 600.

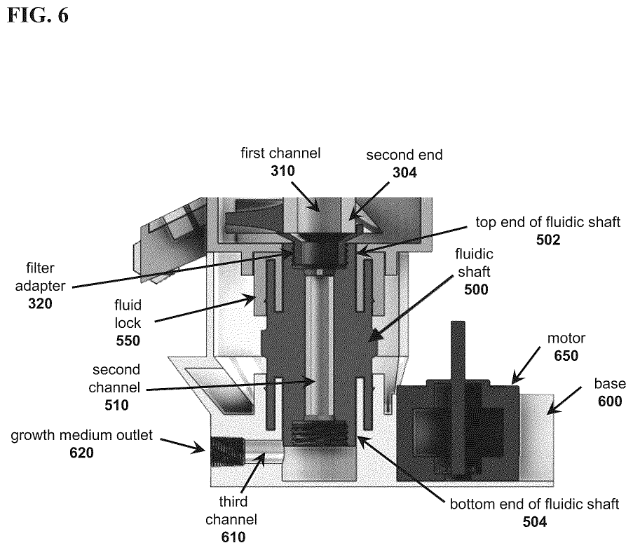

In some embodiments, fluidic shaft 500 is configured to be concentrically aligned with separation chamber 200, filter column 300, and mixing element 400 (FIG. 2 and FIG. 5). In some embodiments, fluidic shaft 500 comprises a substantially cylindrical shape. In some embodiments, fluidic shaft comprises second channel 510 that extends longitudinally through the length of fluidic shaft 500. In some embodiments, fluidic shaft 500 comprises second channel 510 that extends from top end 502 of fluidic shaft 500 to bottom end 504 of fluidic shaft 500 (FIG. 6). In some embodiments, fluidic shaft 500 is cylindrically shaped and comprises a second channel 510 that extends the entire length of fluidic shaft 500 from top end 502 to bottom end 504 of fluidic shaft (FIG. 6). In some embodiments, second channel 510 extends along longitudinal axis 700 (FIG. 2 and FIG. 5).

In some embodiments, fluidic shaft 500 comprises an outer diameter and an inner diameter. In some embodiments, the outer diameter of fluidic shaft 500 is from about 1 cm to about 50 cm (e.g., about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 31 cm, about 32 cm, about 33 cm, about 34 cm, about 35 cm, about 36 cm, about 37 cm, about 38 cm, about 39 cm, about 40 cm, about 41 cm, about 42 cm, about 43 cm, about 44 cm, about 45 cm, about 46 cm, about 47 cm, about 48 cm, about 49 cm, or about 50 cm). In some embodiments, the inner diameter of fluidic shaft 500 is the diameter of second channel 510. In some embodiments, second channel 510 comprises a diameter of from about 0.5 cm to about 48 cm (e.g., about 0.5 cm, about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 31 cm, about 32 cm, about 33 cm, about 34 cm, about 35 cm, about 36 cm, about 37 cm, about 38 cm, about 39 cm, about 40 cm, about 41 cm, about 42 cm, about 43 cm, about 44 cm, about 45 cm, about 46 cm, about 47 cm, or about 48 cm).

In some embodiments, second channel 510 is configured to be in fluid communication with first channel 310. In some embodiments, second channel 510 is configured to be in valved fluid communication with first channel 310. In some embodiments, second channel 510 is configured to be in fluid communication with growth medium outlet 620. In some embodiments, second channel 510 is configured to be in fluid communication with third channel 610 and growth medium outlet 620. In some embodiments, second channel 510 is configured to be in valved fluid communication with growth medium outlet 620. In some embodiments, second channel 510 is configured to be in valved fluid communication with third channel 610 and growth medium outlet 620.

In some embodiments, second end 304 of filter column 300 is configured to be removably coupled to fluidic shaft 500 (FIG. 2). In some embodiments, second end 304 of filter column 300 is configured to be removably coupled to top end 502 of fluidic shaft 500. In some embodiments, second end 304 of filter column 300 is configured to be removably coupled to top end 502 of fluidic shaft 500 by filter adapter 320 (FIG. 6). In some embodiments, bottom end 504 of fluidic shaft 500 is configured to be removably coupled to base 600.

In some embodiments, fluidic shaft 500 is configured to be removably coupled to a fluid lock 550. In some embodiments, top end of fluidic shaft 502 is configured to be removably coupled to fluid lock 550 (FIG. 6). In some embodiments, top end of fluidic shaft 502 is configured to be removably coupled to filter adapter 320 and to fluid lock 550 (FIG. 6). In some embodiments, fluid lock 550 comprises an upper and a lower rotary fluid lock, a belt or a gear (e.g., a bevel, pinion, helical, or miter gear) element to drive rotary motion, and an inner fluid shaft comprising an inner fluid channel. In some embodiments, fluid lock 550 further comprises an upper and lower coupling. In some embodiments, fluid lock 550 further comprises an upper and lower rotary O-ring. In some embodiments, fluid lock 550 further comprises an upper and lower lubricant-filled dynamically sealed fluid lock. In some embodiments, fluid lock 550 further comprises upper and lower bearings. In some embodiments, fluidic shaft 500 and fluid lock 550 are integral. In some embodiments, fluidic shaft 500 and fluid lock are separable. In some embodiments, fluid lock 550 is configured to be rotationally stationary with reference to rotation of fluidic shaft 500. In some embodiments, fluid lock 550 is configured to rotate with fluidic shaft.

In some embodiments, fluidic shaft 500 is configured to concentrically rotate around longitudinal axis 700. In some embodiments, fluidic shaft 500 is configured to concentrically rotate with filter column 300 and mixing element 400 around a longitudinal axis extending from a top end to a bottom end of housing 100. In some embodiments, fluidic shaft 500 is configured to concentrically rotate around longitudinal axis 700 with filter column 300, and mixing element 400.

In some embodiments, rotation of fluidic shaft 500 is configured to be driven by motor 650. In some embodiments, motor 650 is removably coupled to a belt. In some embodiments, the belt couples motor 650 to fluidic shaft 500 such that when motor 650 is engaged the belt is rotated and fluidic shaft 500 is driven to rotate. In some embodiments, motor 650 is configured to drive rotation of fluidic shaft 500 via a belt coupling motor 650 and fluidic shaft 500. In some embodiments, motor 650 is configured to drive rotation of fluidic shaft 500 via direct drive (e.g., motor 650 is operably coupled to fluidic shaft 500), or gears (e.g., bevel, miter, pinion, or regular gears).

In some embodiments, fluidic shaft 500 is configured to rotate around longitudinal axis 700 concentrically with filter column 300 and mixing element 400 in a clockwise direction and/or in a counterclockwise direction. In some embodiments, fluidic shaft 500 is configured to rotate around longitudinal axis 700 concentrically with filter column 300, filter adapter 320, and mixing element 400, in a clockwise direction and/or in a counterclockwise direction. In some embodiments, fluidic shaft 500 is configured to rotate around longitudinal axis 700 concentrically with filter column 300, fluid lock 550, filter adapter 320, and mixing element 400, in a clockwise direction and/or in a counterclockwise direction.

### Base 600

In some embodiments, algae harvesting device comprises base 600. In some embodiments, base 600 is configured to support housing 100, filter column 300, mixing element 400, fluidic shaft 500, fluid lock 550, and motor 650 (FIG. 1). In some embodiments, base 600 is configured to removably couple with bottom end of fluidic shaft 504 (FIG. 6). In some embodiments, base 600 is configured to removably couple with bottom end of fluidic shaft 504 using a snap-fit mechanism, bolts, or pins. In some embodiments, the snap-fit mechanism keeps fluidic lock 550 in place. In some embodiments, a liquid lubricant and/or oil is incorporated between base 600 and bottom end of fluidic shaft 504 as a sealant. In some embodiments, one or more dynamic O-rings are used to hermetically couple base 600 with bottom end of fluidic shaft 504. In some embodiments, one or more dynamic O-rings are used to hermetically couple base 600 with bottom end of fluidic shaft 504 in combination with the liquid lubricant and/or oil.

In some embodiments, base 600 comprises third channel 610. In some embodiments, third channel 610 is configured to fluidly communicate growth medium outlet 620 to second channel 510 (FIG. 2 and FIG. 6). In some embodiments, third channel 610 is configured to be in fluid communication with separation chamber 200, first channel 310, and second channel 510. In some embodiments, base 600 comprises growth medium outlet 620. In some embodiments, base 600 comprises from 1 to 25 (e.g., 1, 2, 3, 4, 5, 6, 7, 8, 9, 10, 11, 12, 13, 14, 15, 16, 17, 18, 19, 20, 21, 22, 23, 24, or 25) growth medium outlet(s) 620. In some embodiments, base 600 comprises from 1 to 10 (e.g., 1, 2, 3, 4, 5, 6, 7, 8, 9, or 10) growth medium outlet(s) 620. In some embodiments, base 600 comprises from 1 to 5 (e.g., 1, 2, 3, 4, or 5) growth medium outlet(s) 620. In some embodiments, third channel 610 is in fluid communication with one or more growth medium outlets.

In some embodiments, the base is configured to align separation chamber 200, filter column 300, the mixing element 400, and the fluidic shaft 500.

In some embodiments, base 600 comprises motor 650. In some embodiments, base 600 is configured to removably couple to motor 650. In some embodiments, motor 650 comprises an electric motor. In some embodiments, the motor 650 comprises a stepper motor, a DC motor, or a servomotor. In some embodiments, the stepper motor is coupled to rotation shaft via a belt. In some embodiments, motor 650 is configured to drive the rotation of filter column 300, mixing element 400, and fluidic shaft 500 around longitudinal axis 700.

### Methods of Harvesting Algae

Herein are provided methods of harvesting algae comprising the steps of providing an algae harvesting device as herein disclosed, driving an algae culture suspension into the one or more inlet(s) of separation chamber 200, rotating fluidic shaft 500 that is coupled to filter column 300 and mixing element 400 using motor 650, driving the algae growth medium through the filter column 300, into first channel 310, through second channel 510, through third channel 610, and through growth medium outlet 620, and driving the algae through the one or more outlet(s) of separation chamber.

In some embodiments, the method of algae harvesting herein disclosed is performed substantially continuously. In some embodiments, the method of harvesting algae herein disclosed is performed substantially continuously for up to 45 days (e.g., for up to 44 days, for up to 43 days, for up to 42 days, for up to 41 days, for up to 40 days, for up to 39 days, for up to 38 days, for up to 37 days, for up to 36 days, for up to 35 days, for up to 34 days, for up to 33 days, for up to 32 days, for up to 31 days, for up to 30 days, for up to 29 days, for up to 28 days, for up to 27 days, for up to 26 days, for up to 25 days, for up to 24 days, for up to 23 days, for up to 22 days, for up to 21 days, for up to 20 days, for up to 19 days, for up to 18 days, for up to 17 days, for up to 16 days, for up to 15 days, for up to 14 days, for up to 13 days, for up to 12 days, for up to 11 days, for up to 10 days, for up to 9 days, for up to 8 days, for up to 7 days, for up to 6 days, for up to 5 days, for up to 4 days, for up to 3 days, or for up to 2 days).

In some embodiments, the method of harvesting algae herein disclosed is performed substantially continuously for up to 30 days (e.g., for up to 30 days, for up to 29 days, for up to 28 days, for up to 27 days, for up to 26 days, for up to 25 days, for up to 24 days, for up to 23 days, for up to 22 days, for up to 21 days, for up to 20 days, for up to 19 days, for up to 18 days, for up to 17 days, for up to 16 days, for up to 15 days, for up to 14 days, for up to 13 days, for up to 12 days, for up to 11 days, for up to 10 days, for up to 9 days, for up to 8 days, for up to 7 days, for up to 6 days, for up to 5 days, for up to 4 days, for up to 3 days, or for up to 2 days).

In some embodiments, the method of harvesting algae herein disclosed is performed continuously for at least half a day (e.g., for at least 1 day, for at least 2 days, for at least 3 days, for at least 4 days, for at least 5 days, for at least 6 days, for at least 7 days, for at least 8 days, for at least 9 days, for at least 10 days, for at least 11 days, for at least 12 days, for at least 13 days, for at least 14 days, for at least 15 days, for at least 16 days, for at least 17 days, for at least 18 days, for at least 19 days, for at least 20 days, for at least 21 days, for at least 22 days, for at least 23 days, for at least 24 days, for at least 25 days, for at least 26 days, for at least 27 days, for at least 28 days, for at least 29 days, or for at least 30 days).

In some embodiments, the method of harvesting algae using the device herein disclosed comprises consuming from about 0 watts to about 500 watts (e.g., about 0 watts to about 400 watts, about 0 watts to about 300 watts, about 0 watts to about 200 watts, about 0 watts to about 100 watts, about 0 watts to about 50 watts, about 0 watts to about 40 watts, about 0 watts to about 30 watts, about 0 watts to about 20 watts, about 0 watts to about 10 watts, about 0 watts to about 5 watts, about 1 watts to about 500 watts, about 5 watts to about 500 watts, about 10 watts to about 500 watts, about 50 watts to about 500 watts, about 100 watts to about 500 watts, about 200 watts to about 500 watts, about 300 watts to about 500 watts, about 400 watts to about 500 watts, about 450 watts to about 500 watts, about 0 watts, about 1 watts, about 2 watts, about 3 watts, about 4 watts, about 5 watts, about 6 watts, about 7 watts, about 8 watts, about 9 watts, about 10 watts, about 25 watts, about 50 watts, about 75 watts, about 100 watts, about 125 watts, about 150 watts, about 175 watts, about 200 watts, about 225 watts, about 250 watts, about 275 watts, about 300 watts, about 325 watts, about 350 watts, about 375 watts, about 400 watts, about 425 watts, about 450 watts, about 475 watts, or about 500 watts) of power per hour.

### Transferring Algae Culture Suspension to the Harvesting Device

In some embodiments, the method of harvesting algae includes driving algae culture suspension into the algae harvesting device. In some embodiments, the method of harvesting algae herein disclosed begins with an algae culture suspension produced outside of the algae harvesting device.

In some embodiments, the algae culture suspension is first driven into the one or more inlet(s) of separation chamber 200 using a positive displacement pump. In some embodiments, the positive displacement pump comprises a peristaltic pump, a gerotor pump, a piston pump, a plunger pump, a diaphragm pump, an internal gear pump, an external gear pump, a lobe pump, a screw pump, or a vane pump. In some embodiments the pump functions with positive pressure (i.e., pushing the algae culture suspension a source located outside of separation chamber into separation chamber 200). In some embodiments, driving the algae culture suspension into separation chamber comprises using negative pressure (i.e., vacuum pressure). In some embodiments, driving the algae culture suspension into separation chamber comprises using a peristaltic pump operably coupled to growth medium outlet 620.

In some embodiments, algae culture suspension is driven into separation chamber at a constant flow rate. In some embodiments, algae culture suspension is driven into separation chamber at variable flow rate. In some embodiments, the flow rate is controlled using an automated process. In some embodiments, algae culture suspension is driven into separation chamber at a volumetric flow rate of from about 0.1 ml/min to about 20,000 ml/min (e.g., about 0.5 ml/min, about 1 ml/min, about 10 ml/min, about 20 ml/min, about 30 ml/min, about 40 ml/min, about 50 ml/min, about 60 ml/min, about 70 ml/min, about 80 ml/min, about 90 ml/min, about 100 ml/min, about 200 ml/min, about 300 ml/min, about 400 ml/min, about 500 ml/min, about 600 ml/min, about 700 ml/min, about 800 ml/min, about 900 ml/min, about 1000 ml/min, about 2000 ml/min, about 3000 ml/min, about 4000 ml/min, about 5000 ml/min, about 6000 ml/min, about 7000 ml/min, about 8000 ml/min, about 9000 ml/min, about 10000 ml/min, about 11000 ml/min, about 12000 ml/min, about 13000 ml/min, about 14000 ml/min, about 15000 ml/min, about 16000 ml/min, about 17000 ml/min, about 18000 ml/min, about 19000 ml/min, or about 20000 ml/min).

Separating Algae from Growth Medium

In some embodiments, the device herein disclosed is useful for separating an algae culture suspension comprising algae microorganisms and algae growth medium into its constituents, a collection of algae microorganisms (e.g., to form of an algae sludge) and sanitized growth medium. In some embodiments, the algae growth medium is driven through the pores of filter column 300 and into first channel 310, separating the algae microorganisms from the algae growth medium.

In some embodiments, separating algae from the algae growth medium includes rotating fluidic shaft 500, which is coupled to filter column 300 and mixing element 400, using motor 650. In some embodiments, the motor is coupled to the fluidic shaft which is itself coupled to filter column 300 and mixing element 400. In some embodiments, rotating fluidic shaft 500, which is connected to filter column 300 and 400, agitates the algae culture suspension inside chamber 200 and generates local vortices near filter column 300, driving the algae growth medium through the pores of the filter column 300 and into first channel 310, into second channel 510, into third channel 610, and out through growth medium outlet 620.

In some embodiments, rotating fluidic shaft 500 connected to filter column 300 and mixing element 400 comprises rotating in a clockwise direction. In some embodiments, rotating fluidic shaft 500 connected to filter column 300 and mixing element 400 comprises rotating in a counterclockwise direction. In some embodiments, rotating fluidic shaft 500 connected to filter column 300 and mixing element 400 comprises rotating at from about 0.1 rpm to about 1000 rpm (e.g., about 0.5 rpm, about 0.75 rpm, about 1 rpm, about 2 rpm, about 5 rpm, about 10 rpm, about 20 rpm, about 30 rpm, about 40 rpm, about 50 rpm, about 60 rpm, about 70 rpm, about 80 rpm, about 90 rpm, about 100 rpm, about 110 rpm, about 120 rpm, about 130 rpm, about 140 rpm, about 150 rpm, about 160 rpm, about 170 rpm, about 180 rpm, about 190 rpm, about 200 rpm, about 210 rpm, about 220 rpm, about 230 rpm, about 240 rpm, about 250 rpm, about 260 rpm, about 270 rpm, about 280 rpm, about 290 rpm, about 300 rpm, about 310 rpm, about 320 rpm, about 330 rpm, about 340 rpm, about 350 rpm, about 360 rpm, about 370 rpm, about 380 rpm, about 390 rpm, about 400 rpm, about 410 rpm, about 420 rpm, about 430 rpm, about 440 rpm, about 450 rpm, about 460 rpm, about 470 rpm, about 480 rpm, about 490 rpm, about 500 rpm, about 510 rpm, about 520 rpm, about 530 rpm, about 540 rpm, about 550 rpm, about 560 rpm, about 570 rpm, about 580 rpm, about 590 rpm, about 600 rpm, about 610 rpm, about 620 rpm, about 630 rpm, about 640 rpm, about 650 rpm, about 660 rpm, about 670 rpm, about 680 rpm, about 690 rpm, about 700 rpm, about 710 rpm, about 720 rpm, about 730 rpm, about 740 rpm, about 750 rpm, about 760 rpm, about 770 rpm, about 780 rpm, about 790 rpm, about 800 rpm, about 810 rpm, about 820 rpm, about 830 rpm, about 840 rpm, about 850 rpm, about 860 rpm, about 870 rpm, about 880 rpm, about 890 rpm, about 900 rpm, about 910 rpm, about 920 rpm, about 930 rpm, about 940 rpm, about 950 rpm, about 960 rpm, about 970 rpm, about 980 rpm, about 990 rpm, or about 1000 rpm). In some embodiments, separating algae from algae growth medium comprises rotating fluidic shaft 500 connected to filter column 300 and mixing element 400 at a constant rotational speed. In some embodiments, separating algae from algae growth medium comprises rotating fluidic shaft 500 connected to filter column 300 and mixing element 400 at different speeds. In some embodiments, the rotational speed of the fluidic shaft is controlled by motor 650. In some embodiments, motor 650 is configured to adjust the rotational speed used for separating algae from algae growth medium in response to a measure of algae produced through the one or more outlet(s) 220 of separation chamber 200. In some embodiments, adjusting rotational speed of at least fluidic shaft 500 connected to filter column 300 and mixing element 400 by motor 650 adjusts an algae production rate (e.g., increasing the rotational speed increases algae production rate and decreasing the rotational speed decreases algae production rate).

In some embodiments, the method of harvesting algae includes separating algae microorganisms from algae growth medium to produce a growth medium comprising at most about 5% w/v (e.g., at most about 4.5%, at most about 4%, at most about 3.5%, at most about 3%, at most about 2.5%, at most about 2%, at most about 1.5%, at most about 1%, at most about 0.5%, at most about 0.4%, at most about 0.3%, at most about 0.2%, at most about 0.1%) of algae microorganisms suspended in the separated growth medium. In some embodiments, the method of harvesting algae includes separating algae microorganisms from algae growth medium to produce a growth medium substantially free of algae microorganisms.

In some embodiments, filter column 300 is configured to be cleaned of algae sludge attached to filter column wall 305 (FIG. 5). In some embodiments, cleaning algae accumulated on filter column wall 305 includes reversing the direction of rotation used for harvesting the algae. In some embodiments, rotating in a substantially reverse direction cleans algae accumulated on filter column wall 305.

Collection of Algae from Separation Chamber

In some embodiments, algae is collected after separating algae from the algae growth medium. In some embodiments, separating algae from the algae growth medium produces a sludge of algae. In some embodiments, the algae (e.g., algae sludge) is driven through the one or more outlet(s) 220 of separation chamber 200 by the rotation of filter column 300 and mixing element 400. In some embodiments, driving the algae through the one or more outlet(s) 220 comprises driving the algae using a peristaltic pump operably coupled to the one or more outlet(s) 200.

In some embodiments, driving the algae through the outlet 220 is performed by the rotation of mixing element 400. In some embodiments, mixing element is shaped to direct algae separated from growth medium (e.g., algae sludge) towards the one or more outlet(s) 220. In some embodiments, mixing element 400 is shaped as a spiral surrounding filter column 300. In some embodiments, the spiral shape of mixing element drives separated algae sludge towards the one or more outlet(s) 220.

In some embodiments, the method of harvesting algae using the device herein disclosed comprises harvesting up to 1 kg (e.g., up to 900 g, up to 850 g, up to 800 g, up to 750 g, up to 700 g, up to 650 g, up to 600 g, up to 550 g, up to 500 g, up to 450 g, up to 400 g, up to 350 g, up to 300 g, up to 250 g, up to 200 g, up to 150 g, up to 100 g, up to 50 g, up to 25 g, or up to 10 g) of algae per day.

### Processing of Growth Medium

In some embodiments, the method of harvesting algae includes the step of sanitizing the growth medium after separating the algae from the algae culture suspension. In some embodiments, the growth medium is driven through the pores of filter column 300, into first channel 310, then into second channel 510, then into third channel 610, and lastly exiting base 600 at growth medium outlet 620. In some embodiments, after growth medium exits growth medium outlet 620, it is processed to remove contaminants (e.g., algae, fungi, bacteria, viruses, and/or protozoa) to produce a sanitized growth medium for reuse in an algae growth and/or cultivation system outside of the harvesting device.

In some embodiments, the growth medium is sanitized after passing through growth medium outlet 620, using ultraviolet radiation, fluid shear stress, heat, or any combination thereof. Any methods of sanitizing fluids using ultraviolet radiation and/or shear stress known in the art can be implemented in the method herein disclosed, such as those described in Li, Shang et al. “Synergetic suppression effects upon the combination of UV-C irradiation and berberine on Microcystis aeruginosa and Scenedesmus obliquus in reclaimed water: Effectiveness and mechanisms.” The Science of the total environment vol. 744 (2020): 140937, and Cai, Shiyu et al. “All Treatment Parameters Affect Environmental Surface Sanitation Efficacy, but Their Relative Importance Depends on the Microbial Target.” Applied and environmental microbiology vol. 87,1 e01748-20. 17 Dec. 2020, which are herein incorporated by reference in their entirety.

### Algae Growth and Cultivation

In some embodiments, the algae harvested using the devices and methods herein disclosed is farmed using aquaculture methods. In some embodiments, the algae are microalgae cultivated in a monoculture (i.e., a culture suspension with algae of a single species). In some embodiments, the algae are microalgae in a polyculture (i.e., a culture with algae of multiple species).

In some embodiments, the algae harvested using the devices and methods herein disclosed can be cultured or grown using open cultivation systems, controlled closed cultivation systems, or any method known in the art, for example as described in Narala et al., Comparison of Microalgae Cultivation in Photobioreactor, Open Raceway Pond, and a Two-Stage Hybrid System, Frontiers in Energy Research, Vol. 4 (2016), herein incorporated by reference in its entirety.

### EXAMPLES

The following examples are provided to illustrate certain disclosed embodiments and are not to be construed as limiting the scope of this disclosure in any way. The present examples provide exemplary methods useful in harvesting algae from locally grown algae cultures.

### Example 1: Harvesting Locally Cultivated Microalgae

A 100-liter suspension of locally cultivated microalgae from an algae cultivation device is processed to harvest the microalgae and separate the microalgae from the algae growth medium of the suspension. The suspension is delivered to an algae harvesting device, with a capacity for processing 5 liters of suspension per hour, by connecting a first peristaltic pump and tubing to the inlet of the separation chamber of the harvesting device. A motor attached to a belt that is attached to the fluidic shaft of the device is activated and rotates the fluidic shaft, the filter column, and the mixing element at 180 rpm. A second peristaltic pump is connected via tubing to the growth medium outlet of the device and is activated. The second peristaltic pump drives the separation of the microalgae suspension being pumped into the separation chamber, leading to accumulation of microalgae sludge on the surface of the rotating filter column, and the crossing of growth medium into the first channel, into the second channel, into the third channel, and out of the growth medium outlet. The rotating mixing element, in the form of a spiral shaped set of blades, directs the microalgae sludge to the outlet of separation chamber to be collected for further processing. The growth medium outlet exiting the growth medium outlet is sanitized by exposing it to 50 W of ultraviolet light of 254 nm wavelength, subsequently the growth medium outlet is collected for use in cultivating more microalgae.

### Exemplary Embodiments

Embodiment 1. In a first aspect, the disclosure provides an algae harvesting device comprising:
a) a housing comprising a separation chamber comprising an inlet, an outlet, and a filter column operably coupled to a mixing element;
b) a fluidic shaft; and c) a base configured to concentrically align the separation chamber, the filter column, the mixing element, and the fluidic shaft, wherein the filter column and mixing element are configured to separate algae from an algae growth medium.

Embodiment 2. The algae harvesting device of embodiment 1, wherein the housing is hermetically sealable.

Embodiment 3. The algae harvesting device of embodiment 1 or embodiment 2, wherein the filter column comprises a first channel extending longitudinally through the length of the filter column.

Embodiment 4. The algae harvesting device of any one of embodiments 1-3, wherein the fluidic shaft comprises a second channel extending longitudinally through the length of the fluidic shaft.

Embodiment 5. The algae harvesting device of any one of embodiments 1-4, wherein the first channel is configured to be in fluid communication with the second channel.

Embodiment 6. The algae harvesting device of any one of embodiments 1-5, wherein the base comprises a third channel configured to fluidly communicate the growth medium outlet to the second channel.

Embodiment 7. The algae harvesting device of any one of embodiments 1-6, wherein the inlet, the separation chamber, the first channel, and the outlet in part (a) are in fluid communication.

Embodiment 8. The algae harvesting device of any one of embodiments 1-7, wherein the filter column comprises a first end at a top end of the separation chamber and a second end at a bottom end of the separation chamber.

Embodiment 9. The algae harvesting device of embodiment 8, wherein the second end of the filter column is configured to be removably coupled to a top end of the fluidic shaft by a filter adapter.

Embodiment 10. The algae harvesting device of any one of embodiments 1-9, wherein the fluidic shaft comprises a bottom end configured to be removably coupled to the base.

Embodiment 11. The algae harvesting device of any one of embodiments 1-10, wherein the mixing element comprises a plurality of blades coupled to the filter column.

Embodiment 12. The algae harvesting device of any one of embodiments 1-11, wherein the filter column comprises a porosity of from about 0.1 to about 0.8.

Embodiment 13. The algae harvesting device of any one of embodiments 1-12, wherein the filter column comprises a pore size of from about 1 μm and 10 μm.

Embodiment 14. The algae harvesting device of any one of embodiments 1-13, wherein the separation chamber comprises a volume of from about 500 ml to about 5000 ml.

Embodiment 15. The algae harvesting device of any one of embodiments 1-14 wherein the filter column, the mixing element, and the fluidic shaft, are configured to concentrically rotate around a longitudinal axis extending from a top end of the housing to the bottom end of the housing.

Embodiment 16. The algae harvesting device of any one of embodiments 1-15, wherein the base further comprises a motor.

Embodiment 17. The algae harvesting device of embodiment 16, wherein the motor is configured to drive the rotation around the longitudinal axis of the filter column, the mixing element, and the fluidic shaft.

Embodiment 18. The algae harvesting device of embodiment 17, wherein the motor comprises an electric motor.

Embodiment 19. The algae harvesting device of embodiment 18, wherein the electric motor comprises a stepper motor, a DC motor, a servomotor.

Embodiment 20. The algae harvesting device of embodiment 19, wherein the stepper motor is coupled to the rotation shaft via a belt.

Embodiment 21. The algae harvesting device of any one of embodiments 1-20, wherein the separation chamber further comprises from 1 to 25 inlets.

Embodiment 22. The algae harvesting device of any one of embodiments 1-21, wherein the separation chamber further comprises from 1 to 25 outlets.

Embodiment 23. The algae harvesting device of any one of embodiments 1-22, wherein the base comprises from 1 to 25 growth medium outlet.

Embodiment 24. The algae harvesting device of any one of embodiments 1-23, wherein the wherein the filter column, the mixing element, and the fluidic shaft, are configured to concentrically rotate around the longitudinal axis in a clockwise direction and/or in a counterclockwise direction.

Embodiment 25. The algae harvesting device of any one of embodiments 1-24, wherein the device is configured to harvest algae with a yield from about 0.1 g to about 1000 g per day.

Embodiment 26. The algae harvesting device of embodiment 11, wherein the plurality of blades is configured to form a helical shape around the filter column.

Embodiment 27. The algae harvesting device of embodiment 26, wherein the plurality of blades is configured to
a) drive the algae growth medium from an algae culture suspension through the filter column, through the first channel, through the second channel, through the third channel, and through the algae growth medium outlet; and
b) drive algae separated from the algae culture suspension from the separation chamber to the outlet.

Embodiment 28. The algae harvesting device of any one of embodiments 1-27, wherein the device is configured to consume power from an off-grid power source.

Embodiment 29. The algae harvesting device of any one of embodiments 1-28, wherein the filter column comprises a sintered filter.

Embodiment 30. A method of harvesting algae comprising:
a) providing an algae harvesting device of any one of embodiments 1-29;
b) driving an algae culture suspension into the inlet of the separation chamber;
c) rotating the fluidic shaft connected to the filter column and the mixing element using the motor;
d) driving the algae growth medium:
   i) through the filter column and into the first channel;
   ii) through the second channel;
   iv) through the third channel; and
   iii) through the growth medium outlet;
and
e) driving the algae through the outlet.

Embodiment 31. The method of embodiment 30, wherein harvesting is performed substantially continuously for up to 45 days.

Embodiment 32. The method of embodiment 30 or 31, wherein harvesting is performed continuously for up to 30 days.

Embodiment 33. The method of any one of embodiments 30-32, wherein step b) comprises driving the algae culture suspension using a positive displacement pump.

Embodiment 34. The method of any one of embodiments 30-33, wherein step b) comprises driving the algae culture suspension using a peristaltic pump connected to the growth medium outlet.

Embodiment 35. The method of any one of embodiments 30-34, wherein the growth medium is sanitized after passing through the growth medium outlet using ultraviolet sterilization and/or shear stress.

Embodiment 36. The method of any one of embodiments 30-35, wherein step e) comprises the using a peristaltic pump connected to the outlet and collecting the algae in a collection chamber.

Embodiment 37. The method of any one of embodiments 30-36, wherein step c) comprises rotating in a clockwise direction.

Embodiment 38. The method of any one of embodiments 30-37, wherein step c) comprises rotating in a counterclockwise direction.

Embodiment 39. The method of any one of embodiments 30-38, further comprising rotating in a substantially reverse direction to clean algae accumulated on the filter column.

Embodiment 40. The method of any one of embodiments 30-39, wherein step c) comprises rotating at from about 0.1 rpm to about 1000 rpm.

Embodiment 41. The method of any one of embodiments 30-40, wherein the method comprises harvesting up to 1 kg of algae per day.

Embodiment 42. The method of any one of embodiments 30-41, wherein the method consumes from about 0 watts to about 500 watts of power per hour.

### OTHER EMBODIMENTS

It will be appreciated that the scope of the present disclosure is to be defined by that which may be understood from the disclosure and claims rather than by the specific embodiments that have been presented by way of example. Elements described with respect to one aspect or embodiment of the present disclosure are also contemplated with respect to other aspects or embodiments of the present disclosure. Throughout the description, where compositions or methods are described as having, including, or comprising specific elements, compositions that consist essentially of, consist of, or do not comprise the recited elements are likewise hereby disclosed. Moreover, recitation of claim elements in connection with a particular independent claim support recitation of such elements in connection with other independent claims. All references cited herein are hereby incorporated by reference.

What is claimed is:

1. An algae harvesting device comprising:
a) a housing comprising a separation chamber comprising an inlet, an outlet, and a filter column operably coupled to a mixing element;
b) a fluidic shaft; and
c) a base configured to concentrically align the separation chamber, the filter column, the mixing element, and the fluidic shaft,
wherein the filter column and mixing element are configured to separate algae from an algae growth medium.

2. The algae harvesting device of claim 1, wherein the housing is hermetically sealable.

3. The algae harvesting device of claim 1, wherein the filter column comprises:
(a) a first channel extending longitudinally through the length of the filter column;
(b) a first end at a top end of the separation chamber and a second end at a bottom end of the separation chamber, optionally wherein the second end of the filter column is configured to be removably coupled to a top end of the fluidic shaft by a filter adapter
(c) a porosity of from about 0.1 to about 0.8;
(d) a pore size of from about 1 μm and 10 μm; or
(e) a sintered filter.

4. The algae harvesting device of claim 1, wherein the fluidic shaft comprises:
(a) a second channel extending longitudinally through the length of the fluidic shaft; and
(b) a bottom end configured to be removably coupled to the base.

5. The algae harvesting device of claim 1, wherein the first channel is configured to be in fluid communication with the second channel.

6. The algae harvesting device of claim 1, wherein the base comprises a third channel configured to fluidly communicate the growth medium outlet to the second channel, optionally wherein the base comprises from 1 to 25 growth medium outlet(s).

7. The algae harvesting device of claim 1, wherein the inlet, the separation chamber, the first channel, and the outlet in part (a) are in fluid communication.

8. The algae harvesting device of claim 1, wherein the mixing element comprises a plurality of blades coupled to the filter column, optionally wherein the plurality of blades is configured to form a helical shape around the filter column.

9. The algae harvesting device of claim 1, wherein the separation chamber comprises a volume of from about 500 ml to about 5000 ml, optionally wherein the separation chamber comprises from 1 to 25 inlets, optionally wherein the separation chamber comprises from 1 to 25 outlets.

10. The algae harvesting device of claim 1, wherein the filter column, the mixing element, and the fluidic shaft, are configured to concentrically rotate around a longitudinal axis extending from a top end of the housing to the bottom end of the housing.

11. The algae harvesting device of claim 1, wherein the base further comprises a motor.

12. The algae harvesting device of claim 11, wherein the motor is configured to drive the rotation around the longitudinal axis of the filter column, the mixing element, and the fluidic shaft.

13. The algae harvesting device of claim 10, wherein the wherein the filter column, the mixing element, and the fluidic shaft, are configured to concentrically rotate around the longitudinal axis in a clockwise direction and/or in a counterclockwise direction.

14. The algae harvesting device of claim 1, wherein the device is configured to harvest algae with a yield from about 0.1 g to about 1000 g per day.

15. The algae harvesting device of claim 8, wherein the plurality of blades is configured to
a) drive the algae growth medium from an algae culture suspension through the filter column, through the first channel, through the second channel, through the third channel, and through the algae growth medium outlet; and
b) drive algae separated from the algae culture suspension from the separation chamber to the outlet.

16. The algae harvesting device of claim 1, wherein the device is configured to consume power from an off-grid power source.

17. A method of harvesting algae comprising:
a) providing an algae harvesting device of claim 1;
b) driving an algae culture suspension into the inlet of the separation chamber;
c) rotating the fluidic shaft connected to the filter column and the mixing element using the motor;
d) driving the algae growth medium:
i) through the filter column and into the first channel;
ii) through the second channel;
iv) through the third channel; and
iii) through the growth medium outlet;
and
e) driving the algae through the outlet.

18. The method of claim 17, wherein harvesting is performed substantially continuously for up to 45 days.

19. The method of claim 17, wherein step b) comprises driving the algae culture suspension using a positive displacement pump.

20. The method of claim 17, wherein step e) comprises using a peristaltic pump connected to the outlet and collecting the algae in a collection chamber.
